(19) United States
(12) Patent Application Publication
(10) Pub. No.: US 2007/0239158 A1
(43) Pub. Date: Oct. 11, 2007

(54) Elastic plates for spinal fixation or stabilization

(75) Inventors: Hai H. Trieu, Cordova, TN (US); Michael C. Sherman, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/400,913

(22) Filed: Apr. 10, 2006

(51) Int. Cl. A61B 17/70; A61B 17/80; A61F 2/44

(52) U.S. Cl. 606/61; 606/69; 623/17.13

(57) ABSTRACT

Plates for use in orthopedic procedures are provided, having in certain embodiments at least a part that is elastic between two attachment parts that may be substantially rigid or elastic. Embodiments in which the attachment parts and the elastic part are of separate materials fixed together and in which they are homogeneous are disclosed. Grommets may be used in connection with the attachment portions. The disclosed plates may be connected to vertebrae by anchors such as bone screws.

The present disclosure relates to devices and implants used in osteosynthesis and other orthopedic surgical procedures. Specifically, the present disclosure contemplates varieties of orthopedic plates having at least a portion being elastic.

Plate members for orthopedic use, including use along one or more vertebrae among other places, can be used for stabilization and/or fixation of bones, bone parts, adjacent tissues or a combination of the same. In trauma cases, for example, a damaged vertebra or vertebral motion segment can be connected to one or more adjacent vertebrae or vertebral motion segments via a plate member so as to limit or substantially eliminate relative motion between the damaged tissue and other tissues. In doing so, further damage can be averted, and setting or other healing of the tissue can occur substantially unimpeded by unwanted motion. As another example, in tumor cases a significant amount of bone or adjacent soft tissues may have to be removed from the spine or other locations, resulting in potentially substantial loss of strength in the tissue. A plate member can be connected to a weakened vertebra (in the spinal example) and to adjacent bone tissue to absorb some or most of the stresses that might otherwise fall on the weakened bone.

Each normal spinal segment undergoes a variety of bending and rotational motions. Plate members that provides support and stabilization to injured or weakened vertebrae or vertebral motion segments can prevent not only the range of motion of a given vertebra or segment, but also a particular type or direction of motion, or indeed substantially any motion at all for the instrumented tissue.

### SUMMARY

The present disclosure provides apparatus, systems, and methods for facilitating bone fixation and/or stabilization, including spinal fixation and/or stabilization.

### BRIEF DESCRIPTION OF THE DRAWINGS

FIG. 1 is a top plan view of an embodiment of a plate member.

FIG. 2 is a side elevational view of the embodiment illustrated in FIG. 1.

FIG. 3 is a cross-sectional view of the embodiment illustrated in FIG. 1, taken along the line 3-3 in FIG. 1 and viewed in the direction of the arrows.

FIG. 4 is a perspective view of the embodiment illustrated in FIG. 1.

FIG. 5 is a schematic view of the embodiment illustrated in FIG. 1 attached to vertebrae.

FIG. 6 is a cross-sectional view of the subject matter illustrated in FIG. 5, taken along the line 6-6 in FIG. 5 and viewed in the direction of the arrows.

FIG. 7 is a top plan view of an embodiment of a plate member.

FIG. 8 is a side view of the embodiment illustrated in FIG. 7.

FIG. 9 is a perspective view of the embodiment illustrated in FIG. 7.

FIG. 10 is a top plan view of an embodiment of a plate member.

FIG. 11 is a cross-sectional view of the subject matter illustrated in FIG. 10, taken along the line 11-11 in FIG. 10 and viewed in the direction of the arrows.

FIG. 12 is a top plan view of an embodiment of a plate member.

FIG. 13 is a cross-sectional view of the subject matter illustrated in FIG. 12, taken along the line 13-13 in FIG. 12 and viewed in the direction of the arrows.

FIG. 14A is a schematic view of an embodiment of a plate member attached to vertebrae with embodiments of inelastic members.

FIG. 14B is a schematic view of an embodiment of a plate member attached to vertebrae with embodiments of inelastic members.

FIG. 14C is a schematic view of an embodiment of a plate member attached to vertebrae with an embodiment of an inelastic member.

FIG. 14D is a schematic view of an embodiment of a plate member attached to vertebrae with an embodiment of an inelastic member.

FIG. 14E is a schematic view of an embodiment of a plate member attached to vertebrae with embodiments of inelastic members.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

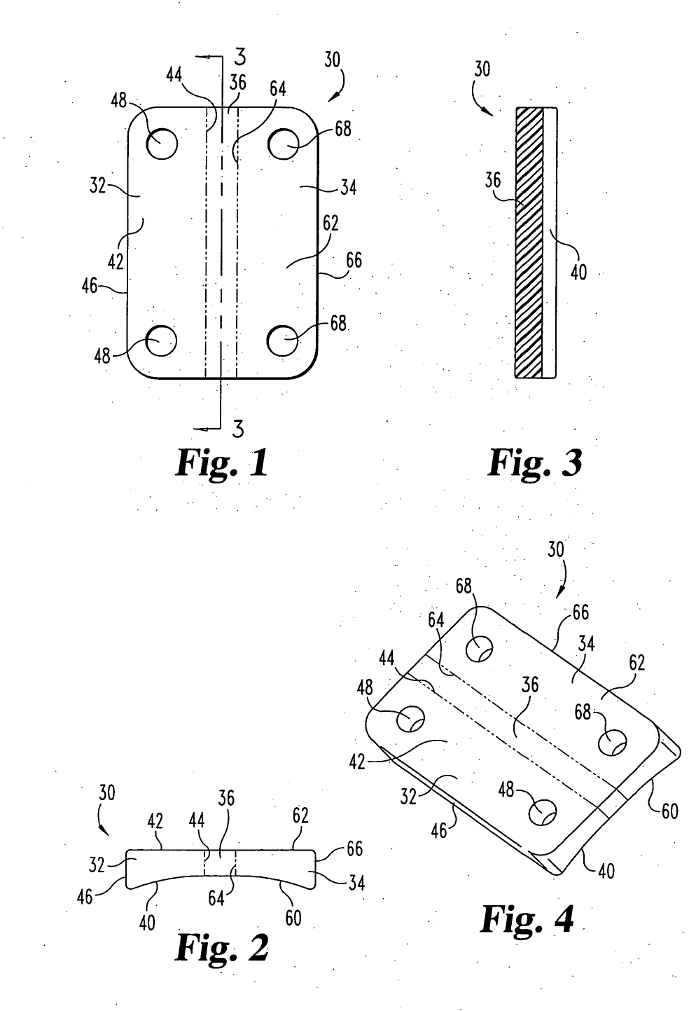

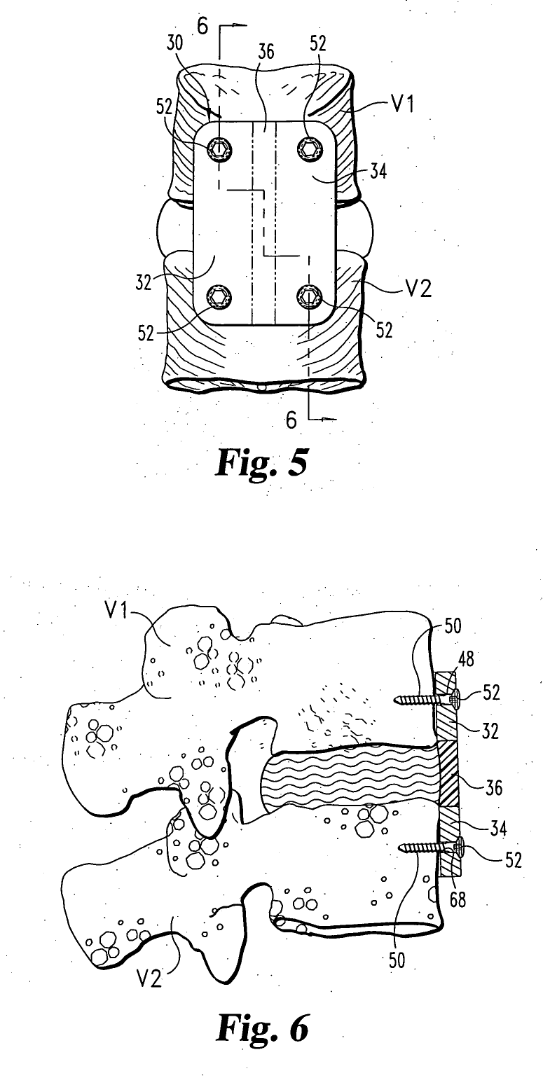

Referring generally to FIGS. 1-6, there is shown an embodiment of a plate member 30. Plate member 30, in that embodiment, is a composite member including a first attachment member or plate portion 32, a second attachment member or plate portion 34, and an elastic medial member 36. Elastic member 36 is firmly attached to the attachment members 32, 34, as further discussed below. In this embodiment, attachment members 32, 34 are engaged to one or more bones, and elastic member 36 provides bendability and support to the instrumented tissue.

Attachment member 32 is substantially rectangular in shape, having a lower surface 40, an upper surface 42 and an internal surface 44. An external surface 46 is also provided in the illustrated embodiment, which in a particular embodiment has a thickness, and in other embodiments may form a sharp or rounded edge. Attachment member 32 has a substantially constant thickness in the illustrated embodiment. Lower surface 40 may be somewhat concave so as to closely mate with a surface of a vertebra, e.g. an anterior vertebral surface, or if attachment member 32 is slim as measured between internal surface 44 and external surface 46, lower surface 40 may be substantially planar. Upper surface 42 may be planar, as seen in the illustrated embodiment, or may be curved in substantially the same manner as lower surface 40.

Two holes 48 are provided that extend through attachment member 32 and between upper surface 42 and lower surface 40. Holes 48 are substantially cylindrical and smooth in the illustrated embodiment, and are sized to accommodate a fixation member 50, such as a bone screw having a head 52 and a threaded shaft 54. Holes 48 may have a diameter that is at least slightly less than the diameter of a head 52 of fixation member 50. Holes 48 may also be provided with a bevel or countersunk surface along upper surface 42 of attachment member 32 so that head 52 can have a lower profile when attaching attachment member 32 to bone. In such an embodiment, head 52 maintains plate member 30 adjacent to or abutting bone tissue, and may press plate member 30 against such tissue.

Attachment member 34 is substantially identical to attachment member 32 in this illustrated embodiment, but is oriented opposite to attachment member 32 so that the two attachment members are essentially mirror images of each other. Thus, attachment member 34 is substantially rectangular in shape, having a lower surface 60, an upper surface 62, an internal surface 64 and an external surface 66, which substantially correspond to surfaces 40, 42, 44 and 46, respectively, described above. In the illustrated embodiment, in which lower surface 60 is somewhat concave so as to closely mate with a surface of a vertebra, the curvature may be correspond to the curvature of lower surface 40 of attachment member 32, so that plate member 30 forms a concave surface that corresponds to a portion of the external surface of one or more vertebrae. As with lower surface 40, lower surface 60 may be substantially planar, particularly in embodiments in which the width of lower surface 60 is small relative to the vertebra(e) to which attachment member 34 is to be attached. Upper surface 62 may be planar, as seen in the illustrated embodiment, or may be curved in substantially the same manner as lower surface 60.

Two holes 68 are provided that extend through attachment member 34 and between upper surface 62 and lower surface 60. Holes 68 are substantially identical to holes 48 in this illustrated embodiment, and thus are sized to accommodate a fixation member 50, such as a bone screw. Holes 68 may also be provided with a bevel or countersunk surface along upper surface 62 of attachment member 34 so that head 52 can have a lower profile when attaching attachment member 34 to bone.

Attachment members 32 and 34 may be made of the same biocompatible material(s), and may be substantially rigid or have some flexibility. Such materials can include polymers such as polyether ether ketone (PEEK), polyether ketone ketone (PEKK), poly-L/D-lactide (PLDLA), polylactic acid (PLA), polyetherterephthalate, polyethylene, polyester, polysulphone, polyesterimide, polyetherimide, polyimide, polypropylene, combinations of them, or others; metals such as titanium, nickel-titanium alloys (e.g. Nitinol), stainless steel or others; and/or ceramics such as calcium phosphate, alumina, zirconia, hydroxyapatite, or others. Combinations of two or more polymers, or of polymer(s) and a metal or ceramic substance, may also be used. Such materials and their formation and physical configuration may be chosen so that attachment members 32 and 34 provide particular support, holding or stiffening characteristics to the overall plate member 30. For example, if relatively thick titanium is used for the manufacture of attachment members 32 and 34, then attachment members 32 and 34 will be substantially rigid and plate member 30 will generally be more stiff and provide stronger support to a vertebra or motion segment. Conversely, if thin metal or somewhat flexible polymers are used, the flexibility inherent in them will provide a plate member 30 that can be more easily manipulated and allows a somewhat greater range of motion to the affected parts of the spine.

Elastic member 36 is attached to each of the attachment members 32 and 34 along their respective internal surfaces 44, 64. In this embodiment, plate member 30 may be compared to a sandwich, with elastic member 36 entirely between attachment members 32 and 34. In this illustrated embodiment, elastic member 36 is relatively thin compared to the width of attachment members 32 and 34, in the illustrated embodiment, and extends or is attached along substantially the entire internal surfaces 44 and/or 64 of attachment members 32 and 34, e.g. the entire length and/or width of internal surfaces 44 and/or 64. Elastic member 36 provides flexibility to plate member 30, and can also provide for storage of tension or potential energy that can be applied against one or both attachment members 32, 34, as further described below. Embodiments of elastic member 36 can be made from polymers such as polyurethane, silicone, silicone-polyurethane copolymer, rubbers such as polyolefin rubbers, hydrogels, combinations of these materials, or other biocompatible materials having elasticity. Such materials may be braided, woven, mesh, porous or other configurations. Elastic member 36 may have a relatively unstressed condition or configuration, to which it will seek to return if stress is placed on it.

Elastic member 36 may be bonded to attachment members 32 and 34 in any of a number of ways. For example, elastic member 36 may be adhesively fixed to attachment members 32 and 34, using a one-part, two-part, pressure sensitive, heat-cured, or other variety of adhesive. As another example, elastic member 36 and/or one or both of attachment members 32 and 34 may be heated or treated with an appropriate solvent to soften or liquefy some of their respective materials, and then attached together. The softened material may intermix, or may enter pores or otherwise interdigitate with another part, and on hardening or curing will form a strong bond between elastic member 36 and attachment members 32 and 34. Similarly, if an attachment member is formed of a three-dimensional fabric of substantially unstretchable material, an elastomeric substance can be injected therein to fill the voids between strands of the fabric, so that on curing the elastomeric material becomes elastic member 36 and is at least partially within or among parts of the inelastic fabric. Elastic member 36 may be mechanically locked together with one or both of attachment members 32 and 34 by overmolding, by providing a dovetail joint (e.g. one or more mortises on either the elastic member 36 or an attachment member 32, and complementary tenon(s) on the other) or other tongue-and-groove-type connection, by laminating elastic member 36 on a surface of one or both attachment members 32 and 34 (e.g. such that elastic member 36 is essentially a sheet atop or underneath attachment members 32 and 34), or by providing hooks or pins on a surface of one or both of attachment members 32 and 34 that penetrate into or through elastic member 36. With respect to lamination, it is observed that when an attachment member (e.g. 32) made of PEEK is laminated with elastomeric material, the elastomeric material becomes embedded in the PEEK material. Other types of firm fixation between elastic member 36 and attachment members 32 and 34 may be used in addition to or in place of the methods and substances described above.

An embodiment of the use of plate member 30 is described below with respect to orthopedic correction or support of a part of the spine. Uses in the cervical, thoracic and lumbar spine, or across one or more of those regions, is contemplated. It will nevertheless be understood that other uses with respect to the spine or other parts of the body are possible.

The illustrated embodiment of plate member 30 is attached to two vertebra V1, V2 that are adjacent or have one or more vertebrae between them, e.g. plate member 30 may span one or more vertebral motion segments. Once the surgeon obtains access to the area to be instrumented, plate member 30 is attached to the vertebrae. In the embodiment in which plate member 30 has holes 48 and fixation members 50 are bone screws, holes may be prepared in each vertebra to which plate member is to be directly attached. Such holes may be drilled, bored or otherwise formed, and may be tapped. Holes 48 of plate member 30 are aligned with the holes in the bore, and anchor members are placed through holes 48 and threaded into the holes in the bone. Head 52 of each fixation member 50 contacts plate member 30 (i.e. one of attachment members 32 and 34) and holds it adjacent to or in contact with the respective vertebra.

Tension or stress can be added to plate member 30 prior to, during or after placement with respect to the vertebrae. Thus, attachment members 32 and 34 can be pushed together, pulled apart, or twisted, or elastic member 36 can be bent around an axis substantially parallel to internal surfaces 44, 64 of attachment members 32, 34. In the illustrated embodiment, an unstressed configuration of elastic member 36 is shown in FIG. 1. Pushing attachment members 32 and 34 toward each other places a compression stress on elastic member 36. As elastic member 36 seeks to regain its unstressed state, it pushes against one or both of attachment members 32 and 34. Conversely, pulling apart attachment members 32 and 34 places elastic member 36 under tension, so that it tends to pull one or both of attachment members 32 and 34 toward the other. Twisting elastic member 36, e.g. by turning one or both of attachment members 32 and 34 with respect to the other around an axis substantially perpendicular to internal surfaces 44 and 64, provides a rotational stress on elastic member 36. Bending elastic member 36 around an axis substantially parallel to internal surfaces 44 and 64 moves lower surfaces 40, 60 or upper surfaces 42, 62 substantially toward each other. Elastic member 36, in that case, has a tension stress on one side and a compression stress on the other. For example, where elastic member 36 is bent so that lower surfaces 40, 60, move substantially toward each other, a compression stress results in a portion of elastic member 36 adjacent lower surfaces 40, 60, and a tension stress results in a portion of elastic member 36 adjacent upper surfaces 42, 62.

Particular stresses or tensions can be placed on plate member 30 such that particular forces are applied to the tissue(s) to which plate member 30 is attached or adjacent. Thus, where at least part of elastic member 36 is under tension or compression, plate member 30 can operate to exert some relative rotational force on respective vertebrae. Where at least part of elastic member 36 is twisted, plate member 30 can operate to exert some relative lateral (e.g. substantially perpendicular to the spinal cord) force on respective vertebrae. Bending stress applied to elastic member 36 can clamp plate member 30 somewhat more rigidly or more loosely to respective vertebrae, as the surgeon may desire.

Plate member 30 can provide the fixation and stabilization of a rigid or flexible plate as well as permit dynamic movement of instrumented vertebrae. Normal motions of vertebrae or vertebral motion segments can be permitted by elastic member 36, and the elasticity of elastic member 36 provides assistance to the vertebrae or segment(s) in returning to the original position. Other procedures can also be performed in conjunction with placement of an embodiment of a plate member such as plate member 30. For example, in the spinal field nucleus replacement, annulus repair, disc replacement, prosthesis or fusion cage placement, or similar procedures or therapeutic steps may be taken. Plate member 30 can then be attached to a first vertebra (either a caudal/superior or a cephalad/inferior vertebra) via one or more anchors such as fixation members 50. If the surgeon desires, an appropriate tension or stress can be placed on plate member 30. Plate member 30 is attached to the second vertebra so that it extends over two or more vertebrae or one or more vertebral motion segments, and lock screws (if needed and provided) can be applied. These steps may be applied in various orders as the surgical situation and/or the surgeon's desire may dictate.

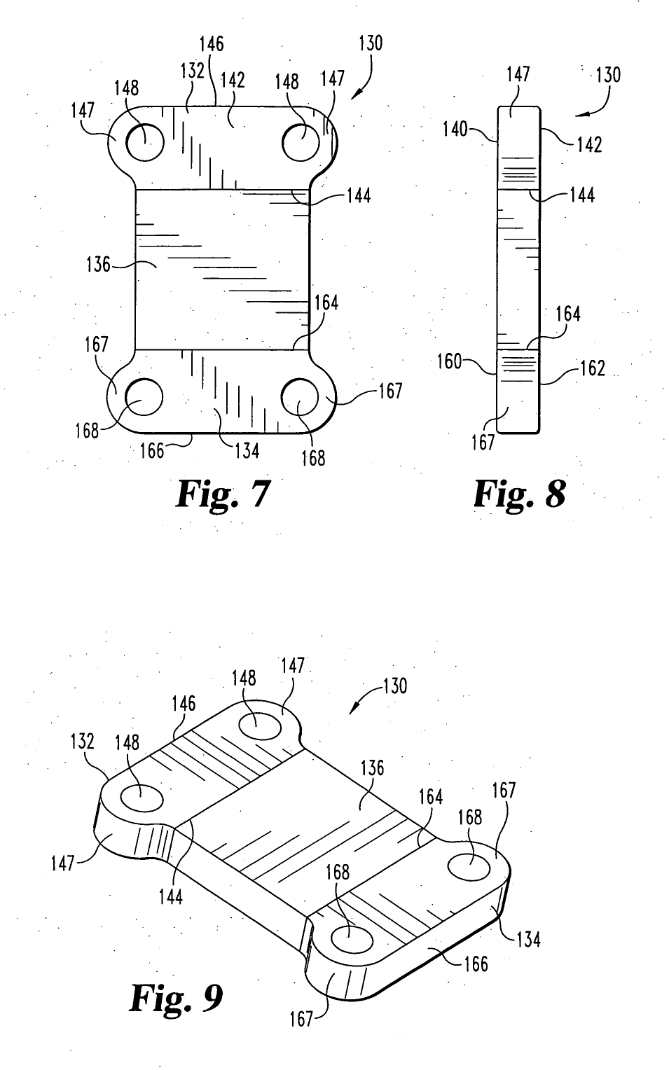

An embodiment of a composite plate member 130, as illustrated in FIGS. 7-9, that is quite similar to the illustrated embodiment of plate member 30 is also shown. Plate member 130 has the same general uses and maneuverabilities as disclosed above with respect to plate member 30. In this illustrated embodiment, plate member 130 includes a first attachment member or plate portion 132, a second attachment member or plate portion 134, and an elastic medial member 136. Elastic member 136 is firmly attached to attachment members 132, 134, as further discussed below. Similar to plate member 30, attachment members 132, 134 are engaged to one or more bones and elastic member 136 provides bendability and support to the instrumented tissue. Attachment members 132 and 134 may be made of the same or similar materials and with the same or similar characteristics as are disclosed above with respect to attachment members 32 and 34.

Attachment member 132 has a roughly rectangular shape in this embodiment, having a lower surface 140, an upper surface 142 and an internal surface 144. An external surface 146 adjacent rounded extensions or ears 147 is also provided in the illustrated embodiment. External surface 146 may have a thickness, or may form a sharp or rounded edge, and in a particular embodiment attachment member 132 has a substantially constant thickness throughout. Lower surface 140 is substantially flat in this illustrated embodiment, or it may be somewhat concave so as to closely mate with a surface of a vertebra, e.g. an anterior vertebral surface. Upper surface 142 is substantially planar in the illustrated embodiment, and in other embodiments may be curved, e.g. in substantially the same manner as an curve in lower surface 140.

Two holes 148 are provided that extend through attachment member 132 and between upper surface 142 and lower surface 140. Holes 148 are substantially cylindrical and smooth in the illustrated embodiment, and are sized to accommodate a fixation member, such as fixation member 50 discussed above. Holes 148 may have a diameter that is at least slightly less than the diameter of head 52 of fixation member 50. Holes 148 may also be provided with a bevel or countersunk surface along upper surface 142 of attachment member 132 so that head 52 can have a lower profile when attaching attachment member 132 to bone. In such an embodiment, head 52 maintains plate member 130 adjacent to or abutting bone tissue, and may press plate member 130 against such tissue.

Attachment member 134 is substantially identical to attachment member 132 in this illustrated embodiment, but is oriented opposite to attachment member 132 so that the two attachment members are essentially mirror images of each other. Thus, attachment member 134 has a generally rectangular shape, a lower surface 160, an upper surface 162, an internal surface 164 and an external surface 166 adjacent extensions or ears 167, which substantially correspond to items 140, 142, 144, 146 and 147, respectively, described above. Two holes 168 are provided that extend through attachment member 134 and between upper surface 162 and lower surface 160. Holes 168 are substantially identical to holes 148 in this illustrated embodiment, and thus are sized to accommodate a fixation member such as fixation member 50, described above. Holes 168 may also be provided with a bevel or countersunk surface along upper surface 162 of attachment member 134.

Elastic member 136 is attached to each of the attachment members 132 and 134 along their respective internal surfaces 144, 164. In this embodiment, attachment members 132 and 134 and elastic member 136 are connected so that attachment members 132 and 134 are generally superior or inferior to the elastic member, as compared to the generally lateral orientation of attachment members 32 and 34 with respect to elastic member 36 of plate member 30. In this illustrated embodiment, elastic member 136 is relatively wide compared to the width of attachment members 132 and 134, e.g. wide enough to extend across one or more disc spaces or motion segments, and extends or is attached along substantially the entire internal surfaces 144 and 164 of attachment members 132 and 134. Elastic member 136 provides flexibility to plate member 130, and can also provide for storage of tension or potential energy that can be applied against one or both attachment members 132, 134, as further described below. Embodiments of elastic member 136 can be made from the same or similar materials and have the same or similar characteristics as those described above with respect to elastic member 36. Further, elastic member 136 may be bonded to attachment members 132 and 134 in any of the ways noted above with respect to the connection of attachment members 32 and 34 to elastic member 36, among others.

It will be observed that in the illustrated embodiment of plate member 130, attachment members 132 and 134 each contact a single vertebra in spinal uses, and elastic member 136 is the only part of plate member 130 that extends across one or more intervertebral disc spaces. In the illustrated embodiment of plate member 30, attachment members 32 and 34 extend across one or more intervertebral disc spaces along with elastic member 36. Accordingly, the illustrated embodiment of plate member 130 may provide a somewhat greater range of motion to instrumented vertebrae, and may be somewhat more effective than plate member 30 at permitting or enabling relative lateral movement of vertebrae or correcting a lateral curvature.

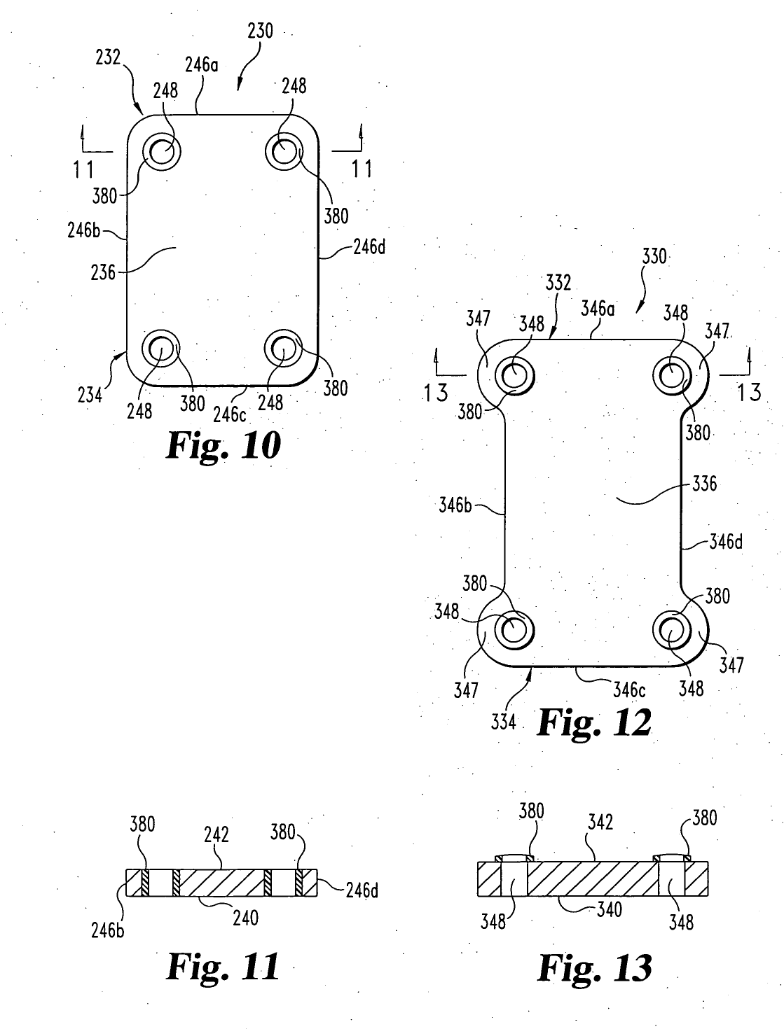

In another embodiment, a plate member 230 (FIG. 10) is shown that is a single piece of elastic material shaped substantially similarly to plate member 30. In comparison with the illustrated embodiment of plate member 30 disclosed above, it may be said that plate member 230 has attachment portions 232 and 234 and a medial portion 236. Plate member 230 may be used in generally the same way for substantially similar purposes to the examples given above with respect to plate members 30 and 130. Further, plate member 230 may be substantially homogeneous in certain embodiments, and may be made of the elastic materials and/or configurations previously described, among others. As with the illustrated embodiment of plate member 30, plate member 230 may be substantially rectangular, having a lower surface 240, an upper surface 242 and external surfaces 246a, 246b, 246c, and 246d. Holes 248 are also provided, which may be substantially the same as holes 48, 68 described above.

An embodiment of a plate member 330, as illustrated in FIG. 12, that has characteristics of the illustrated embodiments of plate members 130 and 230 is also shown. In comparison with the illustrated embodiment of plate member 130 disclosed above, it may be said that plate member 330 has attachment portions 332 and 334 and a medial portion 336. Plate member 330 may be used in generally the same way for substantially similar purposes to the examples given above with respect to plate members 30, 130 and 230. Further, plate member 330 may be substantially homogeneous in certain embodiments, and may be made of the elastic materials and/or configurations previously described, among others. As with the illustrated embodiment of plate member 130, plate member 230 may be roughly rectangular, having a lower surface 340, an upper surface 342 and external surfaces 346a, 346b, 346c, and 346d. Extensions or ears 347 are provided through some or all of which holes 348 extend, which may be substantially the same as holes 48, 68 described above. As above, extensions 347 may be thought of as sticking out from the main part of plate member 330, or medial portion 336 of plate member 330 may be thought of as somewhat narrower than some or all of attachment portions 332 and 334 of plate member 330.

The illustrated embodiments of plate members 230 and 330, as well as embodiments of plate members 30 and 130, may also be used with embodiments of grommets 380. Grommets 380 may be substantially circular and may be substantially continuous or unbroken in circumference or form a split ring. In certain embodiments, grommets 380 may be approximately the same thickness as the plate member with which they are used (e.g. FIG. 11), and in others grommets 380 may be relatively thin as compared to the plate member with which they are used (e.g. FIG. 13). Grommets 380 may be of other shapes or configurations as well. In some embodiments, grommets 380 are made of substantially rigid materials, such as those previously described.

Grommets 380 may be used to provide additional stability or more secure connection between anchors 50 and embodiments of plate members 30, 130, 230 or 330. Accordingly, grommets 380 can be placed substantially within holes in the plate member (e.g. FIGS. 10-11) or on a surface of a plate member adjacent such holes (e.g. FIGS. 12-13). If placed within holes such as holes 248, grommets 380 could be firmly attached to plate member 230 in the ways noted above for attachment of attachment members to elastic members. Grommets 380 can provide a relatively rigid surface against which anchors 50 can press when a plate member such as plate member 230 is attached to bone. Although grommets 380 can be used with any embodiment of plate member illustrated or described herein, and others, they may particularly be used with embodiments such as plate members 230 and 330 that are wholly or substantially made of elastic material.

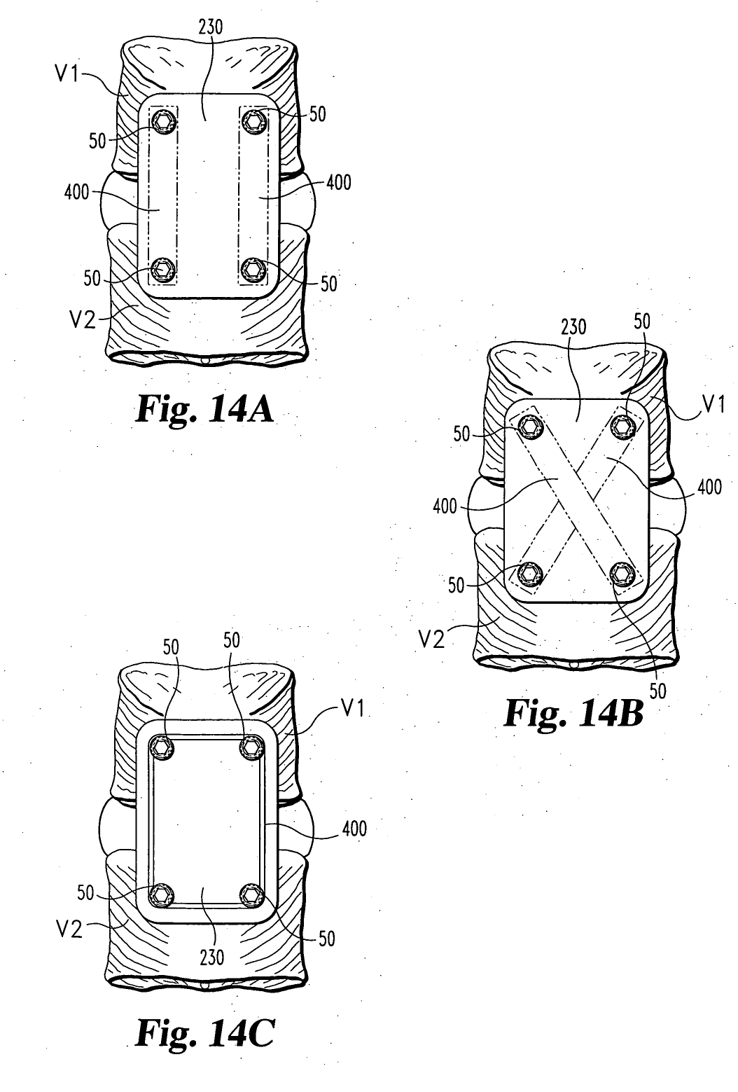

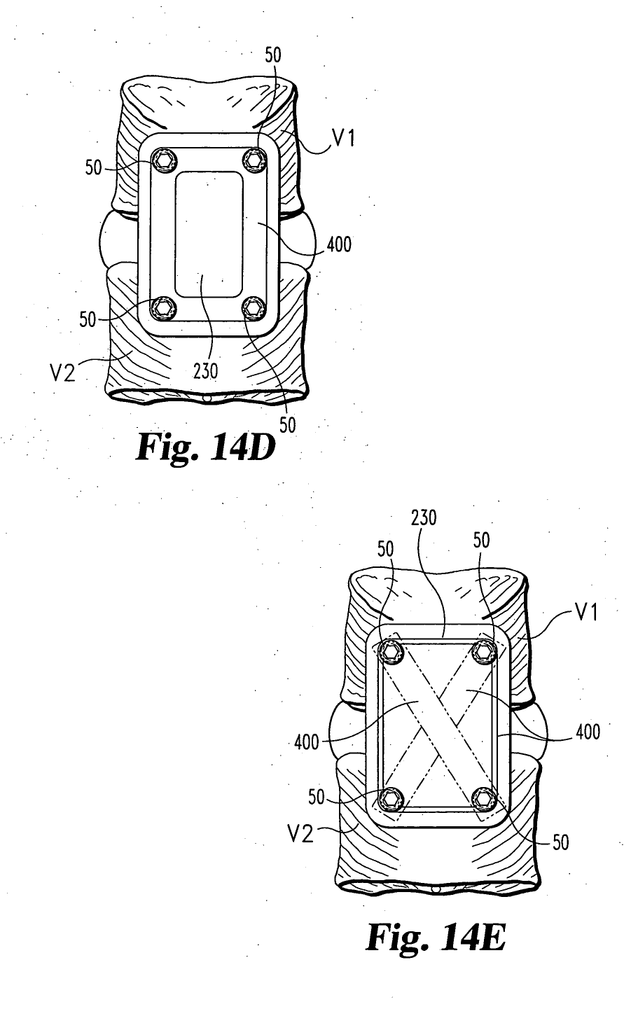

In addition to the above-described devices and methods, elastic and inelastic members can be combined in a plate system in other ways. For instance, a plate member, such as any of plate members 30, 130, 230 or 330 described above, can be combined with external inelastic members. Thus, in embodiments shown in FIGS. 14A-E, a plate member 230 substantially as previously described is attached to adjacent vertebrae V1 and V2. In those embodiments, grommets 380 are not used, although it will be appreciated that they could be used substantially as previously disclosed. To plate member 230 and/or the anchors that hold it to bone tissue, there are attached one or more inelastic members 400, shown schematically in FIGS. 14A-E. Generally, as discussed below, inelastic members 400 are sized, configured and positioned so as to provide a check or limit on the movement of an elastic member such as plate 230.

Inelastic members 400 may be any of a variety of elements that are rigid or flexible, but have a dimensional limit beyond which they are not extendable. As examples, inelastic members 400 could be bars, rods, plates or strips of biocompatible metal, plastic or other sturdy substance, or they could be tethers or cords of polymer, fiber or similar biocompatible flexible substance. In particular embodiments in which inelastic members 400 are flexible cords or tethers, they can be arranged as shown in FIGS. 14A-E. Thus, in the embodiment shown in FIG. 14A, inelastic members 400 are each wrapped around or otherwise securely connected to two fixation members (e.g. members 50 described above) on respective lateral sides of plate member 230. In the embodiment shown in FIG. 14B, inelastic members 400 are each securely connected to two fixation members 50 so that inelastic members 400 cross. In the embodiment shown in FIG. 14C, one inelastic member 400 is securely connected to all four fixation members 50 that attach plate member 230 to bone tissue. Of course, multiple inelastic members 400 could be used in a configuration similar to FIG. 14C. In the embodiment of FIG. 14D, inelastic member 400 has a width great enough to form a layer between a substantial portion or the entirety of the head of fixation member 50 and plate member 230. In such an embodiment, fixation members 50 may pierce or extend through pre-made holes in inelastic member 400. The embodiment of FIG. 14E is essentially a combination of the crossed inelastic members 400 of FIG. 14B and the wrapped inelastic member(s) of FIG. 14C.

In each of these embodiments, both elastic material (in a plate member such as plate member 230) and inelastic material (in member(s) 400) extend across an intervertebral disc space. The inelastic material can take at least a portion of the load provided by the vertebrae, particularly as stress on the elastic member lessens. A crossing pattern, as in FIG. 14B, can provide rotational stability to the joint, while a parallel pattern, as in FIG. 14A, can provide tension stability. A pattern such as that shown in FIG. 14C can provide both tension and rotational stability.

It is to be observed that any type of plate member having at least a part that is elastic can be used in systems and configurations such as are shown in FIGS. 14A-C. Thus, other types of plate members including plate members 30, 130 or 330 disclosed above could be used instead of plate member 230 in such configurations. It is also possible to use plate members in which elastic and inelastic materials are woven or braided together, melded or interembedded with each other, or otherwise firmly interengaged. Such a member could include a rigid or otherwise inelastic frame, such as a wire mesh, in and/or around which elastic material is set. Thus, elastic members such as elastic members 36 and 136 described above could be woven or braided items, made of all elastic or of a combination of elastic and inelastic substances, filaments or fibers, they could be homogeneous masses of elastic material, or they could be a mixture or combination of elastic and inelastic items, as previously indicated.

While the disclosure has been illustrated and described in detail in the drawings and foregoing description, the same is to be considered as illustrative and not restrictive in character, it being understood that only illustrated embodiments have been shown and described and that all changes and modifications that come within the spirit of the disclosure are desired to be protected.

What is claimed is:

1. A composite orthopedic plate, comprising:
a first rigid plate member having at least one opening therethrough;
an elastic member fixed to a portion of said first plate member; and
a second rigid plate member having at least one opening therethrough, said second plate member having a side fixed to said elastic member, wherein said side of said second plate member generally faces said side of said first plate member so that said elastic member is substantially between said plate members, and wherein said elastic member does not overlap at least one of said plate members.

2. The plate of claim 1, wherein said plate members and said elastic member are fixed together so that when said plate member is attached to a portion of the spine, said plate members are substantially lateral of said elastic member.

3. The plate of claim 2, wherein each of said plate members are adapted to be attached to at least two vertebrae.

4. The plate of claim 1, wherein said plate members and said elastic member are fixed together so that when said plate member is attached to a portion of the spine, said first plate member is substantially superior of said elastic member, and said second plate member is substantially inferior of said elastic member.

5. The plate of claim 4, wherein each of said plate members are adapted to be attached to a single vertebra.

6. The plate of claim 1, wherein said plate members are made of at least one of: one or more polymers from the group consisting of polyether ether ketone, polyether ketone ketone, poly-L/D-lactide, polylactic acid, polyethyleneterephthalate, polyethylene, polyester, polysulphone, polyesterimide, polyetherimide, polypropylene, and polyimide; one or more metals or alloys from the group consisting of titanium, nickel-titanium alloys, and stainless steel; and one or more ceramic materials from the group consisting of calcium phosphate and alumina.

7. The plate of claim 1, wherein said elastic member is made of at least one of polyurethane, silicone, silicone-polyurethane copolymer, rubber, hydrogel, and combinations thereof.

8. The plate of claim 1, wherein said elastic member is at least one of homogeneous, woven, braided, and combinations thereof.

9. An orthopedic apparatus, comprising:
an orthopedic plate having a first attachment portion having an upper surface and at least one hole, a second attachment portion having an upper surface and at least one hole, and a medial portion, said medial portion being elastic at least in part;
a grommet defining an aperture, said grommet abutting said plate so that said aperture communicates with one of said holes; and
an anchor for holding said plate with respect to a bone, said anchor extending through said grommet and said hole with which said grommet aperture communicates.

10. The apparatus of claim 9 comprising a plurality of grommets each defining an aperture, each of said grommets abutting said plate so that each of said apertures communicates with a respective one of said holes, and a plurality of anchors for holding said plate with respect to a bone, each of said anchors extending through at least one grommet and said hole with which said grommet aperture communicates.

11. The apparatus of claim 9 wherein said plate is substantially homogeneous.

12. The apparatus of claim 9 wherein said attachment portions are members of a first material, and said medial portion is a member of a second material that is elastic, and said medial portion is fixed to said attachment portions.

13. The apparatus of claim 9 wherein said grommet has a lower surface that abuts one of said upper surfaces.

14. The apparatus of claim 9 wherein said grommet is fixed to said plate within one of said holes of one of said attachment portions.

15. The apparatus of claim 9 wherein at least one of said attachment portions is elastic, and said grommet is substantially rigid, and said grommet abuts said elastic attachment portion.

16. The apparatus of claim 9 wherein said grommet comprises one of a continuous ring and a split ring.

17. A method comprising:
providing an orthopedic plate having a first rigid attachment portion having at least one hole, a second rigid attachment portion having at least one hole, and a medial portion, said medial portion being elastic at least in part, wherein said medial portion does not extend to said holes of said attachment portions;
implanting an intervertebral implant between a first vertebra and a second vertebra;
connecting said first attachment portion to said first vertebra; and
connecting said second attachment portion to said second vertebra.

18. The method of claim 17, wherein said implant is a fusion device, and further comprising placing said elastic medial portion under stress.

19. The method of claim 17 wherein said first vertebra and said second vertebra are adjacent each other.

20. The method of claim 17 wherein at least one additional vertebra is between said first vertebra and said second vertebra.

21. The method of claim 17, wherein said implanting step is substantially all or part of a procedure from the group consisting essentially of nucleus replacement, annulus repair, disc replacement, prosthesis placement, and fusion cage placement.

22. The method of claim 21, wherein said implanting is performed prior to said connecting said first attachment portion.

23. The method of claim 17, wherein said implanting occurs after said connecting said first attachment portion.

24. The method of claim 23, wherein said implanting occurs before said connecting said second attachment portion.
